(19) United States
(12) Patent Application Publication
(10) Pub. No.: US 2005/0022331 A1
(43) Pub. Date: Feb. 3, 2005

(54) Robot cleaner equipped with negative-ion generator

(75) Inventors: Ki-man Kim, Gwangju-city (KR); Jeong-gon Song, Gwangju-city (KR); Yun-sup Hwang, Gwangju-city (KR)

Correspondence Address: BLANK ROME LLP, 600 NEW HAMPSHIRE AVENUE, N.W., WASHINGTON, DC, 20037, US

(73) Assignee: SAMSUNG GWANGJU ELECTRONICS CO., LTD.

(21) Appl. No.: 10/887,918

(22) Filed: Jul. 12, 2004

(30) Foreign Application Priority Data
Jul. 29, 2003 (KR) 2003-52451
May 12, 2004 (KR) 2004-33615

(51) Int. Cl. A47L 5/00

(52) U.S. Cl. 15/319; 15/1.51

(57) ABSTRACT

Disclosed is a robot cleaner which cleans a floor, and generates negative-ions while traveling around a predetermined area. The robot cleaner includes a cleaner body which travels automatically around a cleaning area, a driving unit for driving a plurality of wheels mounted on a lower part of the cleaner body, a suction unit mounted in the cleaner body to draw in dust on a floor, a negative-ion generation unit mounted in the cleaner body to generate a negative-ion, and a control unit. While the robot cleaner travels automatically, it also performs vacuum cleaning using the suction unit, and air cleaning using the negative-ion generation unit, either at the same time or selectively. Accordingly, the floor is cleaned and air is purified by the negative-ion, which enables a hygienic cleansing and a healthy home environment.

### REFERENCE TO RELATED APPLICATION

This application claims priority to Korean Applications No. 2003-52451 filed Jul. 29, 2003, No. 2004-33615 filed May 12, 2004 in the Korean Intellectual Property Office, which is entirely incorporated herein by reference.

### CROSS-REFERENCE TO RELATED APPLICATIONS

This application is related to copending Korean Patent Application Nos. 10-2003-0007-0007426, filed Feb. 6, 2003; 10-2003-0013961, filed Mar. 6, 2003; 10-2003-0029242, filed May 9, 2003; and 10-2003-0050904, filed Jul. 24, 2003, whose disclosures are entirely incorporated herein by reference.

### FIELD OF THE INVENTION

The present invention relates to a robot cleaner equipped with a negative-ion generator, and more particularly to a robot cleaner traveling automatically to clean a cleaning surface, and generating a negative-ion at the same time.

### BACKGROUND OF THE INVENTION

A general robot cleaner performs a cleaning task without requiring a user's intervention by traveling automatically, and drawing in dust on a floor.

The robot cleaner senses distances to obstacles, such as furniture, office appliances, and walls in the cleaning area, by a sensor, and selectively drives a pair of motors therein to prevent colliding with, or being blocked by the obstacles. The robot cleaner alters its direction of motion without assistance during the cleaning task.

Referring to FIG. 1, the robot cleaner includes a cleaner body, a pair of secondary wheels mounted at both sides of a lower front of the cleaner body, and a pair of driving wheels. The driving wheels are mounted at both lower rear sides of the cleaner body. The robot cleaner also includes a pair of motors for rotatably driving the pair of driving wheels, and a timing belt for transmitting a driving force from the rear driving wheels to the front secondary wheels. Further, at a front end of the cleaner body, a suction port is located for drawing in foreign substances such as dust from the cleaning surface. The suction port is driven by a driving motor (not shown).

The above-structured robot cleaner automatically changes the direction of motion by selectively driving the pair of motors. The robot cleaner directs the suction port to clean foreign substances from the cleaning surface. The conventional robot cleaner travels and draws in dust or dirt on the floor through the suction port and discharges filtered air. Therefore, dust not in the immediate cleaning area remains on the cleaning surface. Dust on the floor may fly away and scatter into the air, therefore causing a need of ventilation for a certain time after the cleaning task. In addition, for air cleaning, the user would have to purchase extra ion-generators, at least one for each room. If the user equips each room with the ion-generators, it becomes too wasteful.

Thus, a heretofore unaddressed need exists in the industry to address the aforementioned deficiencies and inadequacies.

### SUMMARY OF THE INVENTION

The present invention overcomes the above-mentioned problems associated with the prior art. Accordingly, it is an aspect of the present invention to provide a robot cleaner capable of automatically traveling around a predetermined area, while performing vacuum and/or air cleaning either at the same time or selectively.

In order to achieve the above-described aspects and features of the present invention, a robot cleaner comprises a cleaner body which automatically travels along a cleaning area, a driving unit to drive a plurality of wheels mounted on a lower part of the cleaner body and a suction unit mounted in the cleaner body to draw in dust on a floor. A negative-ion generation unit is mounted in the cleaner body to generate a negative-ion. A control unit controls the driving unit and directs the robot cleaner according to a pre-stored travel pattern. The control unit also controls operation of the negative-ion generation unit. The robot cleaner moves automatically along the cleaning area, and vacuums using the suction unit while air cleaning via the negative-ion generation unit either, at the same time or selectively.

Preferably, the negative-ion generation unit includes a flow fan, a rotation motor for rotating the flow fan via a power supply and discharging air in the cleaner body. The negative-ion generation unit also includes a discharge duct for discharging air from the cleaner body. A grill member with a plurality of holes is mounted at one end of the discharge duct, and the negative-ion generator is mounted in the grill member to generate negative-ions in the air which are discharged from the discharge duct.

It is also preferable, that the negative-ion generation unit further comprises a plurality of filters for collecting dust in the air, wherein the filtered air is discharged to a predetermined space through a discharge port formed corresponding to a position of the grill member at one side of the body cover. The plurality of filters preferably comprise a first filter for filtering out large-particle dust from the drawn-in air, and a second filter for filtering out fine dust particles and distasteful odors. Preferably, the driving unit comprises a pair of driving motors mounted in the cleaner body driven by a supplied power source, with a pair of driving wheels rotated by the pair of driving motors. A pair of secondary wheels are rotated in accordance with the pair of driving wheels. A driving force transmitting means is responsible for the driving wheels and the secondary wheels to rotate in association with each other. Preferably, the driving force transmitting means is a timing belt.

The grill member is grounded to the cleaner body of the robot cleaner, and may be formed of antistatic resin to avoid being charged with positive electric charge.

Other systems, methods, features and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

These and other features, aspects, and advantages of the present invention will become better understood with regard to the following description, appended claims, and accompanying drawings where:

FIG. 1 is a drawing showing the structure of a bottom of a conventional robot cleaner;

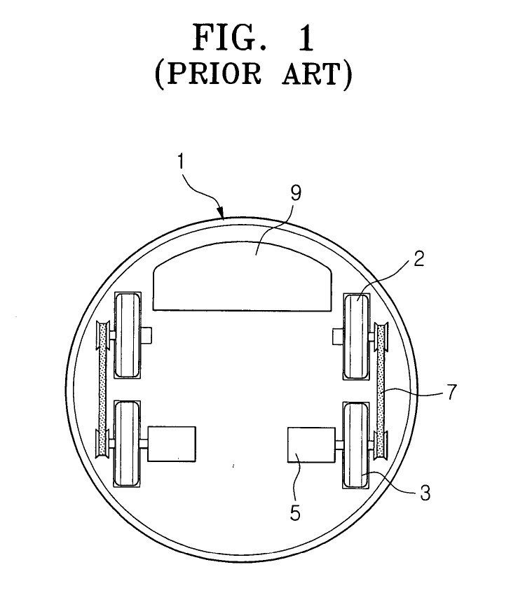

FIG. 2 is a drawing showing a perspective view of a robot cleaner equipped with a negative-ion generator according to the present invention;

FIG. 3 is a block diagram showing a control on the inside of the robot cleaner according to the present invention;

FIG. 4 is a drawing showing an exploded perspective view of main parts of the robot cleaner according to the present invention; and

FIG. 5 is a side view of a robot cleaner having a negative-ion generator being grounded according to an embodiment of the present invention.

### DETAILED DESCRIPTION OF THE PREFERRED EMBODIMENTS

Hereinafter, preferred embodiments of a robot cleaner according to the present invention will be described in detail with reference to the accompanying drawings.

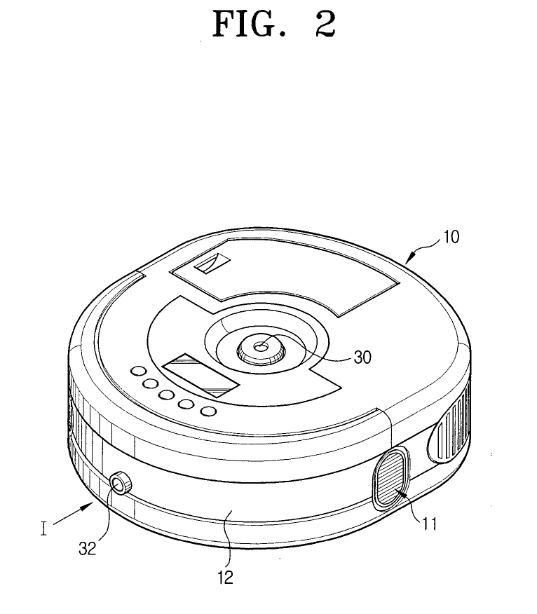

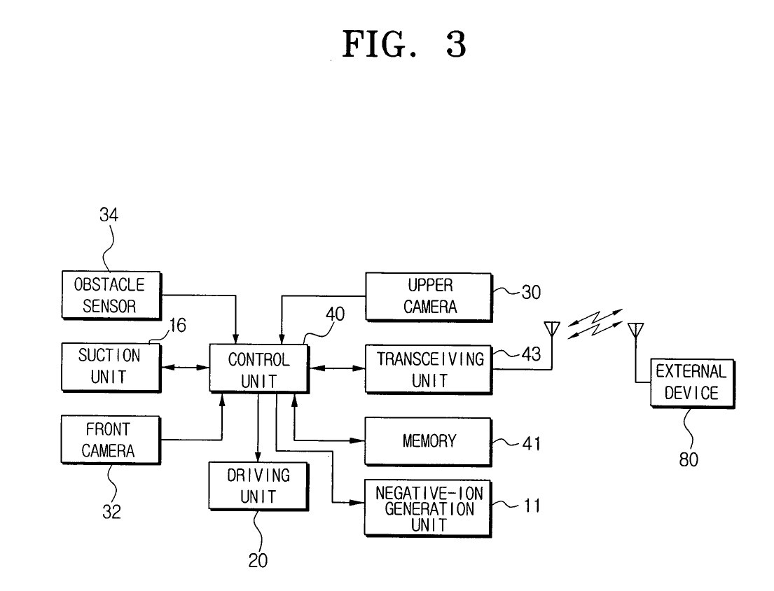

Referring to FIGS. 2-4, the robot cleaner comprises a cleaner body 12, a body cover 14 connected to the cleaner body 12, a suction unit 16, a driving unit 20, an upper camera 30, a front camera 32, an obstacle sensor 34, a control unit 40, a negative-ion generation unit 11, a memory 41, and a transceiving unit 43. A reference symbol ‘I’ represents a front side of the robot cleaner.

The suction unit 16 is mounted on the cleaner body 12 to collect dust on the opposing floor by drawing-in air. The suction unit 16 can be structured according to various well-known methods. For example, the suction unit 16 may comprise a suction motor (not shown) and a dust collecting chamber for collecting dust, which is drawn-in by the suction motor through an inlet or a suction port which is facing the floor.

The driving unit 20 includes a pair of secondary wheels 21 mounted on both front sides, a pair of driving wheels 22 mounted on both rear sides, a pair of driving motors 24 for driving the pair of rear driving wheels 22 respectively, and a driving force transmitting means 25 for transmitting the driving force of the rear driving wheels 22 to the front secondary wheels 21.

In this embodiment, the driving force transmitting means 25 is formed as a timing belt or a gear pulley. In addition, the driving unit 20 rotates the motors 24 independently, clockwise or counterclockwise, according to a control signal from the control unit 40. The robot cleaner's running direction is determined by varying the RPM of the respective driving motors 24.

The front camera 32 is mounted on the cleaner body 12 to photograph an image in front, and output the photographed image to the control unit 40. The upper camera 30 is mounted on the cleaner body 12 to photograph an image of a ceiling, and output the photographed image to the control unit 40. Preferably, a fisheye lens (not shown) is employed for the upper camera 30. The structure of the fisheye lens is disclosed in Korean Patent publication 1996-7005245, Korean Patent publication 1997-48669, and Korean Patent publication 1994-22112. The fisheye lens has been placed on the market by several lens manufacturers. Therefore, detailed description thereof will be omitted.

The obstacle sensors 34 are disposed at a predetermined interval on a circumference of the cleaner body 12 to transmit a signal to the outside and receive a reflected signal. Alternatively, a supersonic wave sensor can be employed for the obstacle sensor 34, which emits a supersonic wave, and receives a reflected supersonic wave. The obstacle sensor 34 is also used for detecting a distance to an obstacle. A rotation sensor can be employed for a running distance sensor (not shown) which is connected to the control unit 40, which detects RPM of the driving wheels 22 or the secondary wheels 21. The rotation sensor can be an encoder which detects the RPM of the respective driving motors 24.

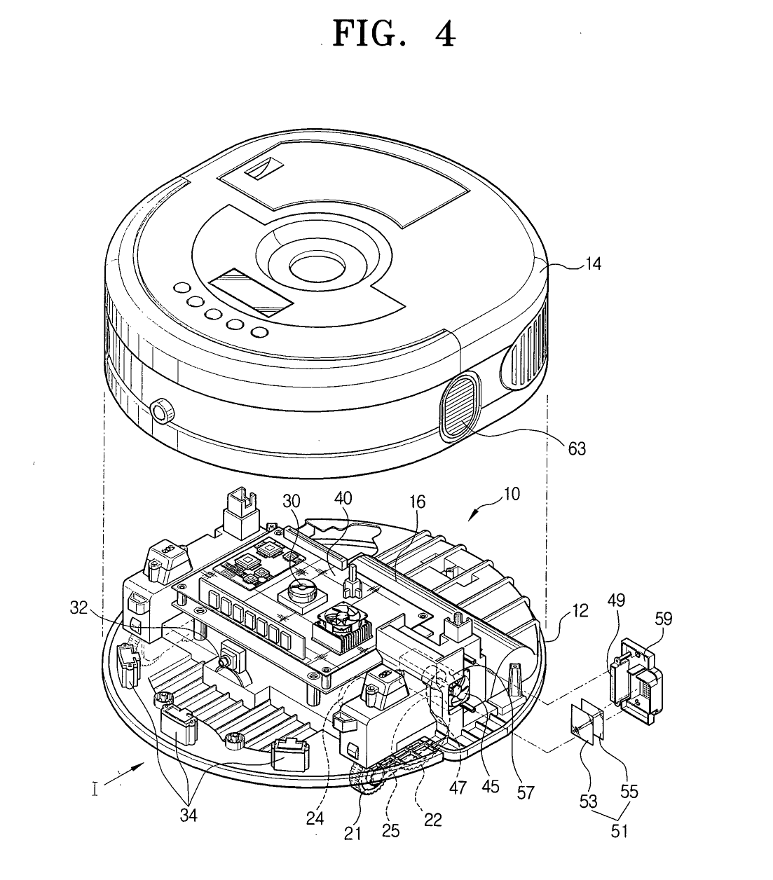

Referring to FIG. 4, the negative-ion generation unit 11 comprises a flow fan 45, a rotation motor 47, a negative-ion generator 49, a discharge duct 57, a grill member 59, and a plurality of filters 51. The flow fan 45 is mounted at one side of the cleaner body 12 to discharge air from inside the cleaner body 12. The rotation motor 47 is powered by a power unit (not shown), and rotates the flow fan 45 to supply a rotational force for discharging air from the cleaner body 12. The negative-ion generator 49 generates a negative-ion from air which is discharged through the flow fan 45. The generated negative-ion is discharged with air, thus cleaning the external air.

A negative-ion includes invisible minute particles which are charged with electricity. An ion is an electrified atom which is a miniscule constituent unit, or an electrified molecule which is an aggregate of atoms. A negative-ion represents an ion which carries a negative charge. When a stable molecule is charged with electricity by specific entities and therefore is electrified, the state of the molecule is called a negative ionization. Oxygen and chlorine are likely to be negatively-ionized. When an electron is bounced from a surface of a substance, an electric emission occurs. A negative-ion generator is a device for ionizing surrounding matters by generating massive electrons based on that principle. Therefore, by supplying a negative voltage of approximately a thousand volts, electrons which carry negative charges are emitted into air at high speed, by a corona discharge, i.e., by a breakage of an insulation in air, with enough energy for ionization to negatively-ionize air.

The negative-ion generator 49 is a commercially available negative-ion generator which generates negative-ions to clean air and provides refreshed air within a certain space. The discharge duct 57 is a discharging path for air in the cleaner body of the robot cleaner. The grill member 59 is connected to an end of the discharge duct 57, with a plurality of holes therein. Air passed through the discharge duct 57 is discharged to a predetermined space through a discharge port 63 which is formed at one side of the body cover 14 which corresponds to a position of the grill member 59.

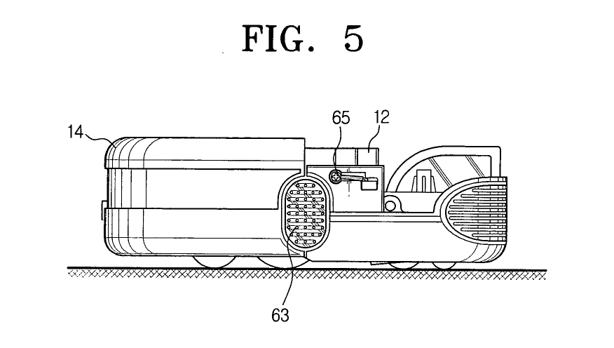

As shown in FIG. 5, the grill member 59 may be grounded to the cleaner body 12 by a grounding unit 65. This is to prevent the negative ion generated by the negative-ion generator 49 from adhering to the grill member 59, when a positive ion is generated at the grill member 59, thereby deteriorating efficiency of generating negative ion. For the same purpose, the discharge port 63 may be formed of antistatic resin as well as the grill member 59 is grounded to the cleaner body 12.

The plurality of filters 51 are mounted at one side of the grill member 59 to filter the air discharged through the discharge duct 57 and include a first filter 53 and a second filter 55. The first filter 53 filters out large-particle dust from the drawn-in air. The second filter 55 filters out fine dust from air particles passed through the first filter 53, and also, deodorizes. Preferably, the second filter 55 is made of a hepa filter to filter out the main causes of respiratory organ disease and allergy, i.e., mold, home dust, animal dander, and virus. Alternatively, the second filter 55 may be a common deodorizing filter. The deodorizing filter purifies air by removing various smells.

The memory 41 stores the image of the ceiling photographed by the upper camera 30, and assists the control unit 40 in calculating location information or running information of the robot cleaner. The transceiving unit 43 sends transmission data to an external device 80 through a transceiver (not shown) mounted in the control unit 40, and transmits a signal from the external device 80 received by the transceiver (not shown) to the control unit 40. The external device 80 is preferably a wireless communication router. The control unit 40 processes the signal received by the transceiving unit 43, and accordingly controls respective parts. In the situation where a key input apparatus (not shown) is disposed in the cleaner body 12 and plural keys equipped therein for setting up functions of the device, the control unit 40 processes key signals inputted from the key input apparatus.

The control unit 40 controls the driving unit 20 to move around a working area according to a predetermined travel pattern, and stores the memory 41 in an image map of the ceiling based on the image photographed by the upper camera 30. Alternatively, upon receiving a wireless command from the key input apparatus or outside, the control unit 40 draws up the image map before the cleaning work. Using the image map while performing the work task, the control unit 40 recognizes a position of the robot cleaner. Upon input from a wireless work request signal from the key input apparatus or from outside, the control unit 40 recognizes the current position of the robot cleaner by comparing the image map with current images inputted from the upper camera 30 and the front camera 32, and directs the driving unit 20 to move from the perceived position which corresponds to a path to the desired destination. The work request signal includes a cleaning task or monitoring through the cameras 30, 32.

While moving along the path to the destination, the control unit 40 calculates a traveling error by using a traveling distance detected by the encoder and the current position which is perceived by comparing the photographed image with the stored image map. The control unit 40 directs the driving unit 15 to track the path to the destination by compensating with the calculated error. While the robot cleaner 10 is operating, control unit 40 operates the suction unit 16 and the negative-ion generation unit 11 according to the work request signal, simultaneously or at the same time. In particular, the flow fan 45 of the negative-ion generation unit 11 is driven by power supplied through the power supplying unit (not shown) of the cleaner body 12. Air discharged through the discharge duct 57 is cleaned through the plurality of filters 51, and upon discharge, cleaned air passes through the negative-ion generator 49. Therefore, ionized air is discharged to a predetermined cleaning area.

In addition, dust or dirt on the floor is vacuumed into the cleaner body 12 through the suction motor (not shown) and a suction pipe, while cleaned air is discharged. As a result, while running along the predetermined area, the robot cleaner cleans the floor discharging cleaned air and the negative-ion to air either selectively or at the same time.

When the user inputs a signal to stop the operation of the driving unit 20 to the external device 80, the robot cleaner 10 remains at a certain position and continues cleaning the floor or generating the negative-ion. Upon completion of the cleaning work or the negative-ion generation, the user inputs a stop command through the external device 80. Accordingly, the control unit 40 of the robot cleaner 10 stops the work task and returns the robot cleaner 10 to an original position. As described above, the robot cleaner 10 equipped with the negative-ion generator, automatically travels along the cleaning area performing vacuum cleaning using the suction unit 16, and air cleaning using the negative-ion generation unit 11, either at the same time or selectively.

As described above, the robot cleaner according to the present invention cleans the floor and generates negative-ions to a predetermined area while traveling automatically along the predetermined area. Accordingly, the robot cleaner is an aid to human health and refreshes a home environment. Further, the robot cleaner according to the present invention, is economical since the user does not have to purchase a separate negative-ion generator, and, convenient to use due to the automatic operation.

Furthermore, The grill member 59 is formed of antistatic resin, and grounded to the cleaner body 12 to keep the grill member 59 in electrically neutral or negative all the time. Accordingly, the negative ion generated by the negative-ion generator 49 is prevented from adhering on a surface of the grill member 59.

While the invention has been shown and described with reference to certain preferred embodiments thereof, it will be understood by those skilled in the art that various changes in form and details may be made therein without departing from the spirit and scope of the invention as defined by the appended claims.

1. A robot cleaner comprising:
a cleaner body which travels automatically along a cleaning area;
a driving unit to drive a plurality of wheels mounted on a lower part of the cleaner body;
a suction unit mounted in the cleaner body to draw in dust on a floor;
a negative-ion generation unit mounted in the cleaner body to generate a negative-ion; and
a control unit to control the driving unit to drive the robot cleaner according to a pre-stored travel pattern, and to control operation of the negative-ion generation unit, wherein
the robot cleaner while running automatically along the cleaning area, performs vacuum cleaning using the suction unit, and air cleaning using the negative-ion generation units at the same time or selectively.

2. The robot cleaner of claim 1, wherein the negative-ion generation unit comprises:
a flow fan;
a rotation motor for rotating the flow fan using a power supply, and discharging air in the cleaner body;
a discharge duct for discharging air from the cleaner body;
a grill member mounted at one end of the discharge duct, and including a plurality of holes; and
a negative-ion generator mounted in the grill member to generate negative-ions in air which is discharged from the discharge duct.

3. The robot cleaner of claim 2, wherein the negative-ion generation unit further comprises a plurality of filters for collecting dust in air, and
filtered air is discharged to a predetermined space through a discharge port formed accordingly to the position of the grill member at one side of the body cover.

4. The robot cleaner of claim 3, wherein the plurality of filters comprise:
a first filter for filtering out large-particle dust from drawn-in air; and
a second filter for filtering out fine dust particles and distasteful odors.

5. The robot cleaner of claim 1, wherein the driving unit comprises:
a pair of driving motors mounted in the cleaner body, and operated by a power source respectively supplied thereto;
a pair of driving wheels rotated by the pair of driving motors;
a pair of secondary wheels rotated in accordance with the pair of driving wheels; and
a driving force transmitting means for causing the driving wheels and the secondary wheels to rotate in association with each other.

6. The robot cleaner of claim 5, wherein the driving force transmitting means is a timing belt.

7. The robot cleaner of claim 1, wherein the grill member is grounded to the cleaner body of the robot cleaner.

8. The robot cleaner of claim 2, wherein the grill member is formed of antistatic resin to avoid being charged with positive electric charge.
